(19) United States
(12) Patent Application Publication
(10) Pub. No.: US 2025/0369938 A1
(43) Pub. Date: Dec. 4, 2025

(54) Water Hardness Test Paper and Preparing Method Thereof

(71) Applicant: Lili Yu, Shenzhen City (CN)

(72) Inventors: Lili Yu, Shenzhen City (CN); Kuizi Wang, Shenzhen City (CN)

(21) Appl. No.: 18/925,089

(22) Filed: Oct. 24, 2024

(30) Foreign Application Priority Data
May 28, 2024 (CN) 2024106745755

(51) Int. Cl. G01N 31/22; G01N 33/18

(52) U.S. Cl. CPC G01N 31/22; G01N 33/1853

(57) ABSTRACT

A water hardness test paper includes a carrier and a water hardness detection substance provided on the carrier, wherein the water hardness detection substance includes a color developer which includes one or more of calcium magnesium reagent, chrome black T, calcium carboxylic acid, azo arsine 1, and azo arsine 3.

### CROSS REFERENCE OF RELATED APPLICATION

This application is a non-provisional application that claims priority under 35 U.S.C. §119 to China application number CN202410674575.5, filing date May 28, 2024, wherein the entire content of which is expressly incorporated herein by reference.

### BACKGROUND OF THE PRESENT INVENTION

### Field of Invention

The present invention relates to the technical field of water test paper, and more particularly to a water hardness test paper and preparing method thereof.

### Description of Related Arts

Currently, the water hardness value of water is different. We should also pay attention to it when choosing drinking water, because they have different effects on the health of our body. Hardness is an important monitoring indicator of water quality. The total hardness of water is about 8, which is more suitable. By monitoring the water hardness value, we can know whether it can be used in industrial production and daily life. For example, water with high hardness can cause soap precipitation and greatly reduce the effectiveness of detergents. In the textile industry, water with too high hardness makes textiles rough and difficult to dye; burning boilers is easy to block pipes and cause boiler explosion accidents; high-hardness water is hard to drink and has a bitter taste. After drinking, it even affects gastrointestinal function; feeding livestock can cause miscarriage in pregnant animals, etc. Since consumers need to frequently test the water hardness value of drinking water to know whether the drinking water used is beneficial to health, there is an urgent need to provide a water hardness test paper and its preparing method on the market for users to detect the water hardness value of drinking water.

### SUMMARY OF THE PRESENT INVENTION

In order to overcome the deficiencies of the prior art, the present invention provides a method for preparing a water hardness test paper, comprising the following steps:

Provide a color developing substance, wherein the color developing substance comprises one or more of calcium magnesium reagent, chrome black T, calcium carboxylic acid, arsenazo 1, and arsenazo 3;

Provide a buffer;

Dissolve the buffer and the color developing substance in pure water, mix to form a first solution;

Add anhydrous ethanol to the first solution, and mix to form a second solution;

Provide a carrier, wherein the carrier is immersed in the second solution;

Take out the carrier from the second solution, and dry the carrier to obtain a water hardness test paper.

As an improvement of the present invention, the buffer comprises one or more of citric acid, sodium citrate, disodium hydrogen phosphate, sodium dihydrogen phosphate, TBS, boric acid, borax, and sodium hydroxide.

As an improvement of the present invention, in the step of providing the color developing substance, wherein the color developing substance comprises one or more of calcium magnesium reagent, chrome black T, calcium carboxylic acid, azo arsenic acid 1, and azo arsenic acid 3. Specifically, the color developing substance comprises one or more of calcium magnesium reagent, chrome black T, calcium carboxylic acid, azo arsenic acid 1, and azo arsenic acid 3, and the amount of the color developing substance in every 100 ml of water is in the range of 0.05 g-6 g.

As an improvement of the present invention, the step of dissolving the buffer and the color developing substance in pure water and mixing to form a first solution is: dissolving the buffer and the color developing substance in pure water, and the amount of the buffer in every 100 ml of water is in the range of 0.1 g-10 g, and mixing to form a first solution.

As an improvement of the present invention, the step of dissolving the buffer and the color developing substance in pure water, the amount of the buffer added in every 100 ml of water is in the range of 0.1 g-10 g, and mixing to form the first solution is: dissolving the buffer and the color developing substance in 500 ml of pure water, the amount of the buffer added in every 100 ml of water is in the range of 0.1 g-10 g, and mixing to form the first solution.

As an improvement of the present invention, the step of adding anhydrous ethanol to the first solution and mixing to form the second solution is: adding 500 ml of anhydrous ethanol to the first solution, and mixing to form the second solution.

As an improvement of the present invention, the step of taking out the carrier from the second solution and drying the carrier to obtain a water hardness test paper is: taking out the carrier from the second solution and drying the carrier at 100 degrees Celsius to obtain the water hardness test paper.

The present invention also provides a water hardness test paper comprising a carrier, wherein the carrier is covered with a water hardness detection substance which is used to detect the water hardness value and develop color, and the carrier is also provided with a filter paper.

As an improvement of the present invention, the area of the filter paper accounts for 4%-7% of the area of the carrier.

As an improvement of the present invention, the width range of the carrier is 3-7 mm, and the length range of the carrier is 60-110 mm.

As an improvement of the present invention, the width range of the filter paper is 3-7 mm, the length range of the filter paper is 3-8 mm, and the thickness of the filter paper is 0.1 mm-0.8 mm.

As an improvement of the present invention, the water hardness detection substance is used to detect the water hardness value and develop color, and the water hardness detection substance is formed by mixing a color developing substance and a buffer.

As an improvement of the present invention, the color developing substance comprises one or more of calcium magnesium reagent, chrome black T, calcium carboxylic acid, azo arsine 1, and azo arsine 3.

As an improvement of the present invention, the buffer comprises one or more of citric acid, sodium citrate, disodium hydrogen phosphate, sodium dihydrogen phosphate, TRIS, boric acid, borax, and sodium hydroxide.

The beneficial effects of the present invention are as follows: through the above steps, the color developing substance is provided, and the color developing substance comprises one or more of calcium magnesium reagent, chrome black T, calcium carboxylic acid, azo arsenic acid 1, and azo arsenic acid 3; a buffer is provided; the buffer and the color developing substance are dissolved in pure water, and mixed to form the first solution; anhydrous ethanol is added to the first solution, and mixed to form the second solution; the carrier is provided, and the carrier is immersed in the second solution; the carrier is taken out from the second solution, and the carrier is dried to obtain the water hardness test paper, so that the user can put the water hardness test paper into the water for detection, and when the color developing substance contacts solutions with different water hardness values, it shows its own structural changes with different colors, and the water hardness value of the current test solution is known by comparing it with the comparison color card. In addition, the buffer can improve the color response of the color developing substance to water hardness, and provide a more stable color response, and the buffer can be complexed with the color developing substance to form a brighter and more gorgeous color, and stabilize the color.

Additional advantages and features of the invention will become apparent from the description which follows, and may be realized by means of the instrumentalities and combinations particularly pointing out in the appended claims.

According to the present invention, the foregoing and other objects and advantages are attained by a water hardness test paper, comprising:
a carrier; and
a water hardness detection substance provided on the carrier, wherein the water hardness detection substance comprises a color developer which comprises one or more of calcium magnesium reagent, chrome black T, calcium carboxylic acid, azo arsine 1, and azo arsine 3.

According to some embodiments, the water hardness detection substance is formed by immersing the carrier in an immersing solution containing the color developer and a buffer which comprises one or more of citric acid, disodium EDTA, sodium citrate, disodium EDTA, disodium hydrogen phosphate, sodium dihydrogen phosphate, TRIS, boric acid, borax, and sodium hydroxide.

According to some embodiments, a container is provided, wherein a plurality of color blocks are painted on an outer surface of the container for indicating levels of water hardness.

According to some embodiments, the plurality of color blocks comprises a first color block, a second color block, a third color block, a fourth color block, a fifth color block, and a sixth color block, wherein the detection result corresponding to the first color block is 0 mg/L, the detection result corresponding to the second color block is 25 mg/L, the detection result corresponding to the third color block is 50 mg/L, the detection result corresponding to the fourth color block is 120 mg/L, and the detection result corresponding to the fifth color block is 250 mg/L, the detection result corresponding to the sixth color block is 425 mg/L.

According to some embodiments, abase layer is provided, wherein the carrier is attached on the base layer. Preferably, the base layer is made of Polyethylene Terephthalate.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

In order to more clearly illustrate the technical solutions in the embodiments of the present invention, the drawings required for use in the description of the embodiments will be briefly introduced below. The drawings in the following description are only some embodiments of the present invention. For ordinary technicians in this field, other drawings can also be obtained based on these drawings without creative work.

The present invention is further described below in conjunction with the drawings and embodiments.

FIG. 1 is a schematic diagram illustrating the preparing method of a water hardness test paper according to a first preferred embodiment of the present invention;

FIG. 2 is another schematic diagram illustrating the preparing method of the water hardness test paper according to the above first preferred embodiment of the present invention;

FIG. 3 is a perspective view of the water hardness test paper according to the above first preferred embodiment of the present invention;

FIG. 4 is a perspective view illustrating a container storing the water hardness test paper of a second preferred embodiment of the present invention.

FIG. 5 is an exploded view illustrating the container storing with the water hardness test paper of the above second preferred embodiment of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

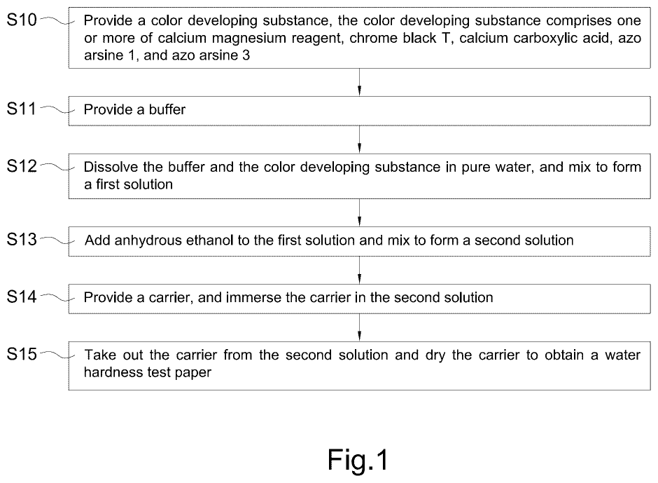

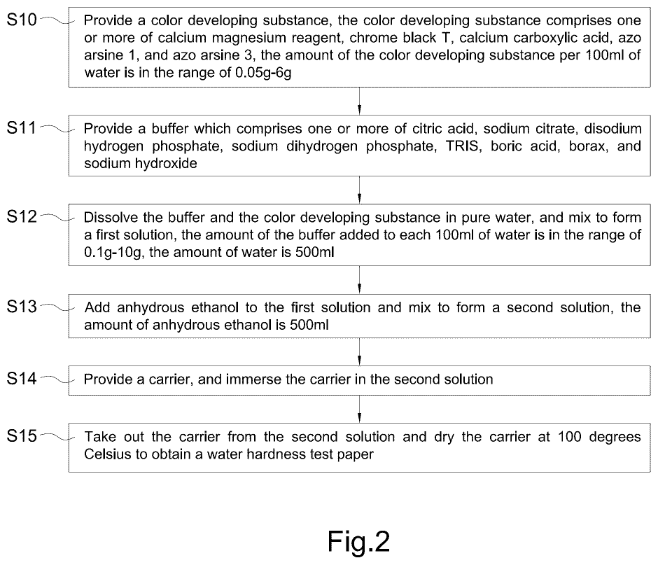

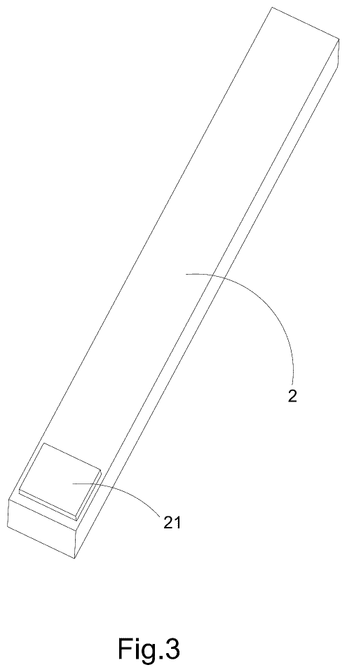

With reference to FIGS. 1 to 3, a method for preparing a water hardness test paper comprises the following steps.

Step S10: Provide a color developing substance, the color developing substance comprises one or more of calcium magnesium reagent, chrome black T, calcium carboxylic acid, azo arsine 1, and azo arsine 3.

In this step, the color developing substance is provided, and the color developing substance comprises one or more of calcium magnesium reagent, chrome black T, calcium carboxylic acid, azo arsine 1, and azo arsine 3, and the amount of the color developing substance per 100 ml of water is in the range of 0.05 g-6 g.

Step S11: Provide a buffer;

In this step, the buffer comprises one or more of citric acid, sodium citrate, disodium hydrogen phosphate, sodium dihydrogen phosphate, TBS (Tris Buffered Saline, trishydroxymethylaminomethane buffered saline), boric acid, borax, and sodium hydroxide. Through the above steps, the buffer can improve the color response of the color developing substance to water hardness and provide a more stable color response, and the buffer can be complexed with the color developing substance to form a brighter and more gorgeous color, and stabilize the color.

Step S12: Dissolve the buffer and the color developing substance in pure water, and mix to form a first solution.

In this step, dissolve the buffer and the color developing substance in pure water, and the amount of the buffer added to each 100 ml of water is in the range of 0.1 g-10 g, and mix to form the first solution; specifically, the buffer and the color developing substance are dissolved in 500 ml of pure water, and the amount of the buffer added to each 100 ml of water is in the range of 0.1 g-10 g, and mix to form the first solution.

Step S13: Add anhydrous ethanol to the first solution and mix to form a second solution; specifically, add 500 ml of anhydrous ethanol to the first solution and mix to form the second solution.

Step S14: Provide a carrier, and immerse the carrier in the second solution.

Step S15: Take out the carrier from the second solution and dry the carrier to obtain a water hardness test paper.

In this step, the carrier is taken out from the second solution, and the carrier is dried at 100 degrees Celsius to obtain a water hardness test paper.

Accordingly, the preparing method comprises the steps of: providing a color developing substance which comprises one or more of calcium magnesium reagent, chrome black T, calcium carboxylic acid, azo arsenic 1, and azo arsenic 3; providing a buffer; dissolving the buffer and the color developing substance in pure water, mixing to form a first solution; adding anhydrous ethanol to the first solution, mixing to form a second solution; providing a carrier, and immersing the carrier in the second solution; taking out the carrier from the second solution, and drying the carrier to obtain a water hardness test paper, so that the user can put the water hardness test paper into the water for detection, and when the color developing substance contacts solutions with different water hardness values, it shows its own structural changes with different colors, and the user is able to know the water hardness value of the current test solution by comparing it with the comparison color card. In addition, the buffer can improve the color response of the color developing substance to water hardness, and provide a more stable color response, and the buffer can be complexed with the color developing substance to form a brighter and more gorgeous color, and stabilize the color.

Referring to FIGS. 1 to 3, a water hardness test paper is illustrated to comprises a carrier 2 which is provided with a water hardness detection substance for detecting the water hardness value and develop color, and a filter paper 21 is also provided on the carrier 2. The area of the filter paper 21 accounts for 4%-7% of the area of the carrier 2. Specifically, the width range of the carrier 2 is 3-7 mm, and the length range of the carrier 2 is 60-110 mm. Further, the width range of the filter paper 21 is 3-7 mm, the length range of the filter paper 21 is 3-8 mm, and the thickness of the filter paper 21 is 0.1 mm-0.8 mm. Further, the water hardness detection layer is used to detect the water hardness value and develop color, and the water hardness detection layer is formed by mixing a color developing substance and a buffer. Further, the color developing substance comprises one or more of calcium magnesium reagent, chrome black T. calcium carboxylic acid, azo arsine 1, and azo arsine 3. Furthermore, the buffer comprises one or more of citric acid, sodium citrate, disodium hydrogen phosphate, sodium dihydrogen phosphate, TRIS, boric acid, borax, and sodium hydroxide.

Accordingly, the preparing method of the water hardness paper comprises the steps of: providing a color developing substance which comprises one or more of calcium magnesium reagent, chrome black T, calcium carboxylic acid, azo arsenic 1, and azo arsenic 3; providing a buffer; dissolving the buffer and the color developing substance in pure water, mixing to form a first solution; adding anhydrous ethanol to the first solution, mixing to form a second solution; providing a carrier, and immersing the carrier in the second solution; taking out the carrier from the second solution, and drying the carrier to obtain the water hardness test paper, so that the user can put the water hardness test paper into the water for detection, and when the color developing substance contacts solutions with different water hardness values, it shows its own structural changes with different colors, and the user is able to know the water hardness value of the current test solution comparing it with the comparison color card. In addition, the buffer can improve the color response of the color developing substance to water hardness, and provide a more stable color response, and the buffer can be complexed with the color developing substance to form a brighter and more gorgeous color, and stabilize the color. Moreover, the dimensions of the carrier and the filter paper are reasonably designed, which is convenient for the production and packaging of water hardness test paper, and the color development effect is better.

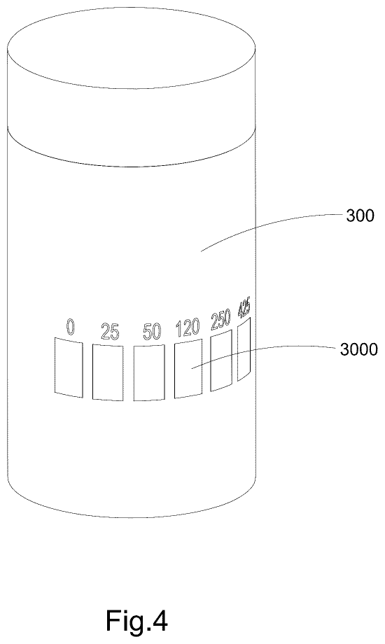

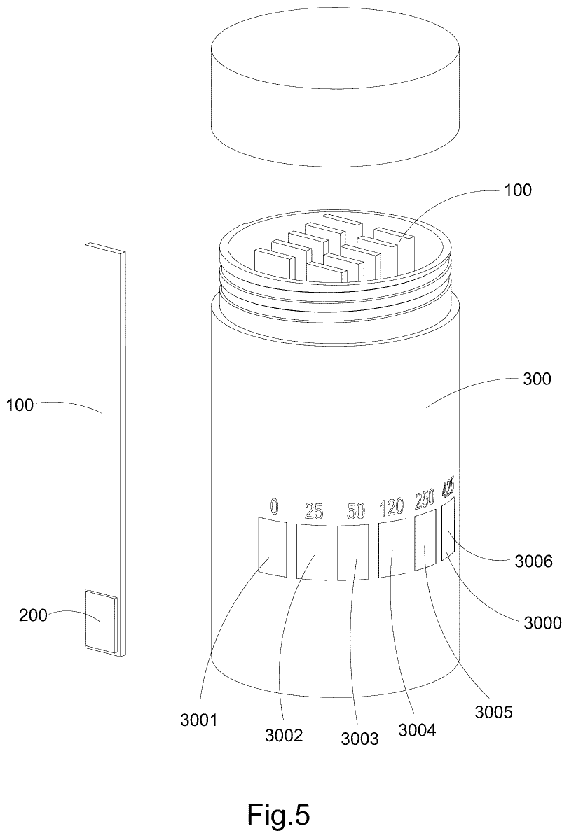

Referring to FIGS. 4 to 5 of the drawings, a water hardness test paper according to a second preferred embodiment of the present invention is illustrated. The water hardness test paper comprises a base layer 100, and a carrier 200 attached to the base layer 100 for carrying a water hardness detection substance.

Water hardness generally refers to the concentration of calcium and magnesium ions present in the water, and it is a crucial indicator of water quality. The hardness level of water can significantly affect both industrial processes and personal health, making it important to monitor.

The water hardness test paper of the present invention can be applied into various occasions. It can ensures that the hardness of drinking water remains within safe and acceptable levels to avoid potential health issues such as gastrointestinal discomfort or kidney problems. Homeowners and consumers can use the water hardness test paper to monitor their drinking water and maintain health. Detecting hard water is important for protecting home appliances such as water heaters, washing machines, and dishwashers from limescale buildup, which can reduce their efficiency and lifespan. In addition, hard water affects the performance of soaps and detergents, leading to reduced cleaning efficiency in laundry and dishwashing. Regular testing helps adjust water softeners.

Monitoring water hardness is critical to prevent scale formation in industrial boilers and heating systems, which can lead to energy inefficiency, damage, and accidents. Soft water is essential in the textile industry for processes like washing, dyeing, and finishing to prevent fabric stiffness and ensure even color distribution. Consistent water hardness levels are necessary for maintaining product quality in brewing, dairy production, and food processing. Water hardness can also impact soil quality and plant health, especially in regions with hard water, making it important for farmers to monitor the water used in irrigation. High-hardness water can affect animal health, particularly in dairy and poultry farming, leading to decreased productivity and increased health risks. Hardness affects the pH balance and mineral content of water in aquariums and fish farms, influencing the health and growth of fish and aquatic plants

In pharmaceutical manufacturing, water with controlled hardness is necessary to ensure the purity and effectiveness of medications. Water used in dialysis must have low hardness to prevent harm to patients, making constant monitoring essential.

The base layer 100 is made of a polymer material such as PET (Polyethylene Terephthalate), PEN (Polyethylene Naphthalate), PP (Polypropylene), and PE (Polyethylene). In this embodiment, the base layer 100 is made of PET.

PET is known for its excellent mechanical strength, providing a robust and durable base layer that can withstand handling and environmental stress during use and storage. PET exhibits strong resistance to chemicals, including acids and bases. This characteristic ensures that the base layer 100 does not degrade or react when exposed to various substances, preserving the integrity of the hardness detection test paper.

PET has low shrinkage and maintains its dimensions under different environmental conditions, such as changes in temperature and humidity. This stability is crucial for the consistent performance of the test paper. PET also offers excellent barrier properties against moisture and gases, protecting the water hardness detection substance from premature degradation due to exposure to air or moisture. This extends the shelf life and reliability of the test paper.

PET can be widely used in manufacturing due to its ease of processing. It can be easily extruded, laminated, or coated, making it a versatile material for creating a consistent and high-quality base layer 100.

The carrier 200 is used for being immersed in the second solution to carry the water hardness detection substance. In this embodiment, the carrier 200 is made of a filtering paper. Accordingly, the filtering paper is designed to absorb liquids efficiently, ensuring that the immersing second solution, which contains the water hardness detection substance, is evenly distributed across the carrier 200. This uniform absorption is crucial for consistent test results.

The filtering paper has a porous structure, which allows it to hold and distribute the water hardness detection substance effectively. The porosity ensures that the test paper has sufficient surface area for the calcium and magnesium ions in the water to react with the detection substance, leading to a more accurate color change. The filtering paper is flexible and easy to handle, making it ideal for manufacturing processes where the carrier needs to be immersed, dried, and further processed. Its flexibility also allows it to conform to different shapes or sizes as needed.

Since filtering paper is chemically inert and does not react with the water hardness detection substance, it minimizes any potential interference in the color reaction, ensuring that the test results are reliable and accurate.

As a biodegradable material, the filtering paper also contributes to the environmental sustainability of the product, particularly when compared to synthetic alternatives. This can be an important factor for consumers and industries focused on eco-friendly solutions.

The water hardness detection substance on the carrier 200 comprises one or more of calcium magnesium reagent, chrome black T, calcium carboxylic acid, azo arsine 1, and azo arsine 3.

The calcium magnesium reagent is specifically formulated to detect the presence of both calcium and magnesium ions in water. These ions form complexes with the reagent, resulting in a color change that indicates hardness levels. It can simultaneously detect both calcium and magnesium ions, the primary causes of water hardness. It is beneficial in that it provides a clear color change, making it easy to visually assess water hardness without the need for complex equipment.

Chrome Black T is a complexometric indicator used to detect metal ions like calcium and magnesium. When used in water hardness detection, it changes color in the presence of these ions. It is very sensitive to calcium and magnesium ions, allowing for accurate detection even in low concentrations.

Calcium carboxylic acid reacts with calcium ions to form insoluble calcium salts, which can be detected through precipitation or color changes.

Azo arsine 1 is a reagent that reacts with metal ions, including calcium and magnesium, resulting in a color change that indicates the hardness level of the water. It can detect a broad range of metal ions, making it useful in testing various types of water with different hardness compositions. It also provides a strong and visible color change, making it easy to interpret results without specialized instrument.

Azo arsine 3, like Azo arsine 1, is used for detecting metal ions in water by producing a color change upon reaction with calcium and magnesium ions.

The buffer comprises one or more of citric acid, disodium EDTA, sodium citrate, disodium EDTA, disodium hydrogen phosphate, sodium dihydrogen phosphate, TRIS, boric acid, borax, and sodium hydroxide. The buffer in the water hardness detection system is critical for maintaining the pH at an optimal level for the reactions between the hardness detection reagents and the calcium and magnesium ions.

Accordingly, the buffer helps maintain a stable pH environment that is crucial for the optimal performance of the color developing substances. Color developing reactions, such as those involving calcium and magnesium reagents, are often highly pH-sensitive.

By stabilizing the pH at the optimal level for the specific reagents, the buffer ensures that the color developing substance reacts more sensitively to changes in water hardness. This leads to a quicker and more noticeable color change, improving the detection process.

Without a stable buffer, fluctuations in pH could lead to inconsistent color responses, making it difficult to accurately assess the hardness. The buffer minimizes this issue, leading to more reliable and reproducible results.

The buffer also ensures that the color developing reaction proceeds smoothly, maintaining consistent reaction conditions throughout the test. This results in a more stable and lasting color response.

Accordingly, a well-buffered system prevents the color from fading prematurely, allowing the test paper to display the results over a longer period. This stability is important for the user to interpret the results accurately, especially when immediate comparison to a color chart is required.

In addition, external factors like temperature and small impurities in the water can affect the pH. The buffer system helps mitigate these influences, ensuring the color response remains stable and reliable across different environments.

Furthermore, the buffer can also influence the intensity and brightness of the color produced by the reaction. It interacts with the color developing substances to create a clearer, more distinguishable color change. A brighter and more vibrant color makes the result easier to interpret by the naked eye. This is especially useful for test strips designed for consumer use, where precise interpretation of color change is key to assessing water hardness.

The buffer may complex with the color developing substance, producing colors that are not only brighter but also more visually appealing. This is particularly advantageous in tests where different levels of water hardness are represented by different color intensities, as it allows for better differentiation between hardness levels.

It is worth mentioning that the buffer also helps stabilize the color produced by the reaction, preventing color shifts or degradation over time. This ensures the color remains true to the actual hardness level for the duration of the test. Stabilizing the color ensures that the user can accurately interpret the test result without worrying about the color changing after the initial reaction. This is critical for consistency, especially when the test strip needs to be read after a set period. Stabilizing the color response also ensures that the test strips maintain their efficacy for longer periods, making them more reliable and extending their shelf life.

Citric acid is a weak organic acid commonly used in buffering systems to control the pH. It can be employed in the present invention to stabilize the pH in the slightly acidic range, which is necessary for hardness detection reagents to function properly. Citric acid can also chelate metal ions, helping to control their concentration in water samples and allowing for more accurate detection of calcium and magnesium.

Sodium citrate is the sodium salt of citric acid and is commonly used as a buffering agent to maintain a stable pH. It can work well with citric acid to create a buffer system that can maintain a consistent pH level. Sodium citrate does not interfere with the calcium and magnesium ions, allowing accurate hardness detection in the present invention.

Disodium EDTA can effectively bind with calcium and magnesium ions, which are the main contributors to water hardness. This chelation forms stable complexes with these ions, facilitating their detection in water samples. The ability of disodium EDTA to complex with these ions improves the precision and accuracy of the hardness measurement, ensuring a clear response from the color-developing substances used in the detection system.

Disodium hydrogen phosphate is a phosphate buffer that helps control pH in the slightly alkaline range. It can keep the pH stable in a slightly basic range, which is suitable for certain reagents like Chrome Black T to perform effectively. It also does not react with calcium or magnesium, preserving the integrity of hardness detection.

Sodium dihydrogen phosphate is another phosphate salt used for buffering in combination with disodium hydrogen phosphate. When used with disodium hydrogen phosphate, it creates a buffer system that can control pH in both acidic and slightly alkaline environments. It can allow for precise pH control across a broad pH range, accommodating different types of hardness detection reagents.

TRIS is a versatile buffering agent widely used in biochemical applications, especially for maintaining a stable pH in a slightly alkaline environment. It can be effective at maintaining pH around neutral or slightly alkaline, which is ideal for many water hardness detection reactions. It does not interfere with calcium or magnesium detection, ensuring accurate results.

Boric acid is a weak acid commonly used in buffering solutions to maintain a stable pH in mildly acidic conditions. It provides a gentle pH adjustment, which can be beneficial when detection reagents are sensitive to large pH fluctuations. Its use in water testing is well-established, making it reliable and effective.

Borax, or sodium tetraborate, is often used with boric acid to create a borate buffer system, providing pH stability in mildly alkaline conditions. Borax, when combined with boric acid, helps maintain a stable pH in the alkaline range, which is ideal for some of the water hardness detection reagents. It stabilizes the pH without interacting with hardness ions, preserving the integrity of the water hardness test.

Sodium hydroxide is a strong base used to adjust the pH to alkaline levels when necessary. It can quickly and effectively raise pH levels, allowing for rapid adjustment in cases where the water sample is too acidic.

The present invention further provides a container 300 for storing a plurality of the water hardness test paper, and a plurality of color blocks 3000 are painted on an outer surface of the container 300 for indicating the levels of water hardness.

In this embodiment, the plurality of color blocks 3000 comprises a first color block 3001, a second color block 3002, a third color block 3003, a fourth color block 3004, a fifth color block 3005, and a sixth color block 3006. The detection result corresponding to the first color block 3001 is 0 mg/L, the detection result corresponding to the second color block 3002 is 25 mg/L, the detection result corresponding to the third color block 3003 is 50 mg/L, the detection result corresponding to the fourth color block 3004 is 120 mg/L, and the detection result corresponding to the fifth color block 3005 is 250 mg/L, the detection result corresponding to the sixth color block 3006 is 425 mg/L.

The inclusion of color blocks 300 directly on the container 300 provides an easy and immediate reference for users to compare the color change of the test paper with the standard color blocks. This feature simplifies the process of interpreting test results, as users can directly compare the test paper to the color blocks on the container without needing a separate reference card.

The color blocks 3000 being painted on the container 300 ensure that the reference guide is always available with the test papers. This design reduces the risk of misplacing or losing the color reference, making the test kit more user-friendly and accessible, especially in field conditions.

By incorporating a range of color blocks 3000 that correspond to specific water hardness concentration levels, the design offers clear visual differentiation between different concentrations. This clarity helps users more accurately determine water hardness levels, even in situations where precise readings are critical.

Since the color blocks 3000 are painted on the container 300, they are less likely to be damaged or worn compared to a separate paper reference card. This increases the durability of the test kit, ensuring that the reference guide remains intact and legible over time. The color blocks 300 may be painted on an outer surface of the container 300, or the color blocks 300 are painted on a paper sheet and the paper sheet is then attached on the container body of the container 300.

Combining the test paper storage and the color reference in one container makes the entire testing kit more compact and portable. Users can carry the container with them easily, knowing that they have everything needed for water hardness testing in one convenient package.

The present invention further provides a method for preparing the water hardness test paper, and the method comprises the steps of providing an immersing solution and immersing the carrier 200 in the immersing solution.

In the step of providing the immersing solution, the buffer and the color developing substance are dissolved and mixed in pure water, and then anhydrous ethanol is added to obtain the immersing solution. In this step, the buffer and the color developing substance are dissolved in 500 ml of pure water, and the amount of the buffer added to each 100 ml of water is in the range of 0.1 g-10 g, the amount of the color developing substance added to each 100 ml of water is in the range of 0.05 g-6 g. Accordingly, 0.5 g-50 g buffer and 0.25 g-30 g color developing substance are added in the 500 ml of water. And then, 500 ml of anhydrous ethanol is added to the mixture to obtain the immersing solution.

In the step of immersing the carrier 200 in the immersing solution, the carrier is immersed in the above immersing solution and then is taken out from the immersing solution and dried to obtain the water hardness test paper.

In the manufacturing process of the water hardness test paper, an integral piece of the carriers 200 is immersed into the above immersing solution and dried, and then is cut into small pieces and a piece of the carrier 200 can be attached to the base layer 100 to form the final product of the water hardness test paper.

As a first example, 0.35 g calcium magnesium reagent, 0.7 g citric acid, 0.423 g disodium EDTA and 2.8 g sodium hydroxide are added and mixed in 500 ml water, and then 500 ml of anhydrous ethanol is added to the mixture to obtain the immersing solution. Under alkaline conditions, the calcium magnesium reagent undergoes a complexation reaction with calcium and magnesium ions to display a pink color.

As a second example, 0.462 g Chrome Black T, 1.01 g disodium hydrogen phosphate, and 1.41 g sodium dihydrogen phosphate are dissolved and mixed in 500 ml water, and then 500 ml of anhydrous ethanol is added to the mixture to obtain the immersing solution. When Chrome Black T binds to calcium or magnesium ions in hard water, it changes to a red or wine color, signaling the presence of hardness causing ions. In the absence of calcium and magnesium ions, Chrome Black T typically displays a blue or purple color.

As a third example, 0.862 g calcium carboxylic acid, 1.42 g citric acid and 0.526 g sodium citrate are added and mixed in 500 ml water, and then 500 ml of anhydrous ethanol is added to the mixture to obtain the immersing solution. When used in the detection of water hardness, calcium carboxylic acids typically form insoluble precipitates when they encounter high concentrations of calcium or magnesium ions. This leads to visual changes, as calcium carboxylic acids may result in cloudiness, precipitate formation, or other physical changes that can indicate water hardness.

As a fourth example, 0.348 g azo arsenic acid 1, 0.76 g boric acid, and 4.93 g borax are added and mixed in 500 ml water, and then 500 ml of anhydrous ethanol is added to the mixture to obtain the immersing solution. When Azo Arsenic Acid 1 is used in water hardness detection, it typically forms a complex with calcium or magnesium ions, which results in a color change. The color displayed by this reagent can vary depending on the concentration of calcium or magnesium ions present in the water. In low concentration of Calcium/Magnesium Ions, a light pink can be displayed while in a high concentration of Calcium/Magnesium Ions, upon binding with calcium or magnesium ions, Azo Arsenic Acid 1 typically displays a purple or violet color.

The above is one or more implementation modes provided in combination with the specific content, and it is not intended that the specific implementation of the present invention is limited to these descriptions. Any method, structure, etc. similar to or identical to the method, structure, etc. of the present invention, or a number of technical deductions or substitutions made on the premise of the concept of the present invention, shall be regarded as the scope of protection of the present invention.

One skilled in the art will understand that the embodiment of the present invention as shown in the drawings and described above is exemplary only and not intended to be limiting. It will thus be seen that the objects of the present invention have been fully and effectively accomplished. The embodiments have been shown and described for the purposes of illustrating the functional and structural principles of the present invention and are subject to change without departure from such principles. Therefore, this invention includes all modifications encompassed within the spirit and scope of the following claims.

1. A water hardness test paper, comprising:
a carrier; and
a water hardness detection substance provided on the carrier, wherein the water hardness detection substance comprises a color developer which comprises one or more of calcium magnesium reagent, chrome black T, calcium carboxylic acid, azo arsine 1, and azo arsine 3.

2. The water hardness test paper according to claim 1, wherein the water hardness detection substance is formed by immersing the carrier in an immersing solution containing the color developer and a buffer which comprises one or more of citric acid, disodium EDTA, sodium citrate, disodium EDTA, disodium hydrogen phosphate, sodium dihydrogen phosphate, TRIS, boric acid, borax, and sodium hydroxide.

3. The water hardness test paper according to claim 1, further comprising a container, wherein a plurality of color blocks are painted on an outer surface of the container for indicating levels of water hardness.

4. The chlorine detection test paper according to claim 2, further comprising a container, wherein a plurality of color blocks are painted on an outer surface of the container for indicating levels of water hardness.

5. The chlorine detection test paper according to claim 4, wherein the plurality of color blocks comprises a first color block, a second color block, a third color block, a fourth color block, a fifth color block, and a sixth color block, wherein the detection result corresponding to the first color block is 0 mg/L, the detection result corresponding to the second color block is 25 mg/L, the detection result corresponding to the third color block is 50 mg/L, the detection result corresponding to the fourth color block is 120 mg/L, and the detection result corresponding to the fifth color block is 250 mg/L, the detection result corresponding to the sixth color block is 425 mg/L.

6. The chlorine detection test paper, as recited in claim 1, further comprising a base layer, wherein the carrier is attached on the base layer.

7. The chlorine detection test paper, as recited in claim 2, further comprising a base layer, wherein the carrier is attached on the base layer which is made of Polyethylene Terephthalate.

8. A preparing method of a water hardness test paper, comprising the following steps:
providing a color developing substance, wherein the color developing substance comprises calcium carboxylic acid, wherein the calcium carboxylic acid reacts with calcium ions to form insoluble calcium salts to be detected through precipitation or color changes;
providing a buffer;
forming a first solution by dissolving the buffer and the color developing substance in pure water;
forming a second solution by adding anhydrous ethanol to the first solution;
providing a carrier and immersing the carrier in the second solution; and
removing the carrier from the second solution and drying the carrier to obtain a water hardness test paper.

9. The method according to claim 8, wherein the buffer comprises one or more of citric acid, sodium citrate, disodium hydrogen phosphate, sodium dihydrogen phosphate, TBS, boric acid, and borax.

10. The method according to claim 8, wherein an amount of the color developing substance in every 100 ml of pure water is in a range of 0.05 g-6 g.

11. The method according to claim 8, wherein an amount of the buffer in every 100 ml of pure water is in a range of 0.1 g-10 g.

12. The method according to claim 9, wherein an amount of the buffer in every 100 ml of pure water is in a range of 0.1 g-10 g.

13. The method according to claim 10, wherein an amount of the buffer in every 100 ml of pure water is in a range of 0.1 g-10 g.

14. The method according to claim 9, wherein the buffer and the color developing substance are dissolved and mixed in 500 ml of pure water to form the first solution, and 500 ml of anhydrous ethanol is added to the first solution to form the second solution, such that a volume ratio between pure water and anhydrous ethanol is 1:1.

15. The method according to claim 13, wherein the buffer and the color developing substance are dissolved and mixed in 500 ml of pure water to form the first solution, and 500 ml of anhydrous ethanol is added to the first solution to form the second solution, such that a volume ratio between pure water and anhydrous ethanol is 1:1.

16. The method according to claim 13, wherein the carrier is dried at 100 degrees Celsius to obtain the water hardness test paper.

17-20. (canceled)

21. The method, as recited in claim 8, wherein the carrier is made of porous material and is provided on a base layer which is made of Polyethylene Terephthalate.

22. The method, as recited in claim 8, further comprising a step of:
providing a plurality of color blocks for indicating levels of water hardness when the water hardness test paper is in contact with water to be tested;
the color blocks comprises a first color block, a second color block, a third color block, a fourth color block, a fifth color block, and a sixth color block, wherein a detection result corresponding to the first color block is 0 mg/L, a detection result corresponding to the second color block is 25 mg/L, a detection result corresponding to the third color block is 50 mg/L, a detection result corresponding to the fourth color block is 120 mg/L, a detection result corresponding to the fifth color block is 250 mg/L, and a detection result corresponding to the sixth color block is 425 mg/L.

23. The method, as recited in claim 22, wherein the color blocks are painted on an outer surface of a container while the water hardness test paper is stored in the container before use.
